(19) United States
(12) Patent Application Publication
(10) Pub. No.: US 2023/0176036 A1
(43) Pub. Date: Jun. 8, 2023

(54) Detection of viruses in facemasks

(71) Applicant: Camas Incorporated, Le Center, MN (US)

(72) Inventors: Scott Simplot, Boise, ID (US); Bradley M. Mitteness, Ghent, MN (US)

(21) Appl. No.: 17/922,520

(22) PCT Filed: Apr. 27, 2021
(86) PCT No.: PCT/US2021/029384

§ 371 Date: Oct. 31, 2022

Related U.S. Application Data
(60) Provisional application No. 63/018,127, filed on Apr. 30, 2020.

(51) Int. Cl. G01N 33/497; G01N 33/569; G01N 21/78

(52) U.S. Cl. CPC G01N 33/497; G01N 33/56983; G01N 21/78; G01N 2333/165

(57) ABSTRACT

Facemasks with an integrated vims detection system are described. The virus detection system is embedded in or on a acemask worn by an individual susceptible to a viral infection. The individual wears a facemask for protective purposes during work or leisure. If the individual develops a viral infection and begins discharging viral particles from mouth and/or nose, the vims detection system embedded in the facemask retains the virus particles. The virus particles are detected in an immunoassay. The virus particles in the virus detection system embedded in the facemask are detected by avian antibodies. The virus detection system in the facemask is configured to detect one or more viruses, for example, SARS-CoV-2.

### FIELD

The present description provides an assay for the detection of viruses in individuals, particularly coronavirus in individuals.

### BACKGROUND

Severe acute respiratory syndrome coronavirus-2 (SARS-CoV-2) is the virus strain that causes coronavirus disease 2019 (COVID-19), a respiratory illness. SARS-CoV-2 is a positive-sense single-stranded RNA virus.

Transmission of SARS-CoV-2 can occur by human-to-human transmission and thus humans in constrained spaces can lead to increase in transmission rates. Transmission occurs primarily via respiratory droplets from coughs and sneezes within a range of about 1.8 meters (6 ft). The virus is also transmitted as aerosol particles. Indirect contact via a contaminated surface is another possible cause of infection. Preliminary research indicates that the virus may remain viable on plastic and steel for up to three days, but does not survive on cardboard for more than one day or on copper for more than four hours; the virus is inactivated by soap, which destabilizes its lipid bilayer.

### SUMMARY

In one aspect, the present description relates to a facemask for an individual comprising a virus detection system, wherein the virus detection system includes a test region. The virus detection system also includes virus binding molecules in the test region. The test region is positioned in the facemask to receive respiratory droplets and/or aerosol particles discharged from the individual. The virus detection system is configured to detect the presence of one or more viruses. The virus detection system may further include one or more detection reagents to provide a signal when the virus is bound to the virus binding particles. The virus detection system may be an enzyme immunoassay (EIA) test. The virus binding molecules may be antibodies. The virus binding molecules may be avian antibodies. The virus detection system may be a direct detection system or an indirect detection system. The virus detection system may be a competitive assay or a sandwich assay. The virus may be a coronavirus. The virus may be severe acute respiratory syndrome coronavirus-2 (SARS-CoV-2). The virus detection system may detect Covid-19. The virus binding molecules may bind the spike protein of SARS-CoV-2. The one or more detection reagents may be selected from the group consisting of enzymes, reporter groups, substrates, cofactors, inhibitors, dyes, radionuclides, luminescent groups, fluorescent groups, colorimetric indicators, and other detecting molecules. The virus detection system may include one or more monoclonal antibodies and/or one or more polyclonal antibodies.

In another aspect, the present description relates to a method of detecting a viral infection in an individual. The method can include analyzing the viral infection status in the individual through a virus detection system embedded in a facemask. The individual may wear the facemask for a time period, wherein the test region is placed to receive the respiratory droplets and/or aerosol particles discharged from the individual. The virus detection system may include a test region and virus binding molecules, wherein the virus detection system is configured to detect one or more viruses. The time period may be at least five minutes. The time period may be between about five minutes and 10 hours. The virus detection system may further include one or more detection reagents. The individual may wear the facemask during an activity. The virus detection system may provide a positive indicator signal when the individual is infected with the virus. The positive indicator signal may be present upon removal of the facemask if the individual is infected with the virus. The positive indicator signal may be present upon addition of one or more detection reagents after the removal of the facemask if the individual is infected with the virus. The method may include performing an EIA test. The method may include virus binding molecules that are antibodies. The virus binding molecules may be avian antibodies. The method may include performing a competitive assay or a sandwich assay. The method may include detecting a coronavirus. The method may include detecting a severe acute respiratory syndrome coronavirus-2 (SARS-Cody-2). The method may detect Covid-19. The virus binding particles may bind the spike protein of SARS-CoV-2. The method may include using one or more detection reagents selected from the group consisting of enzymes, reporter groups, substrates, cofactors, inhibitors, dyes, radionuclides, luminescent groups, fluorescent groups, colorimetric indicators and other detecting molecules. The method may include one or more monoclonal antibodies and/or one or more polyclonal antibodies.

In a further aspect, the present description relates to a method of operating a workplace. The method includes providing a facemask comprising a viral detection system, wherein individuals in the workplace wear the face mask during all or part of the work day and wherein the viral detection system in the facemask is evaluated for the presence of the virus after a time period, wherein the virus detection system is configured to detect one or more viruses. The workplace may be a meat processing factory.

### Definitions

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers to one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The term “and/or” means one or all of the listed elements or a combination of any two or more of the listed elements.

The terms “comprises,” “comprising,” and variations thereof are to be construed as open ended— i.e., additional elements or steps are optional and may or may not be present.

The term “facemask” as referred to herein relates to a facemask that includes a virus detection system. The virus detection system may be integrated into the facemask in a variety of ways as described herein.

The term “respiratory droplets and/or aerosolized particles” as referred to herein include any and all discharge from the mouth and nose of an individual including saliva and the like.

The term “discharged particles” and “discharged viral particles” as referred to herein includes any discharge from an individual that could potentially carry pathogenic organisms, e.g. viral particles. The discharged particles can include respiratory droplets and/or aerosolized particles. These terms will e used interchangeably herein.

The term “spike protein” as referred to herein refers to all or a portion of the spike protein and can include the S1 and/or S2 subunits of the SARS-CoV-2 virus. The term “virus binding molecules” as referred to herein relates to molecules that bind the virus particles. The virus binding molecules can bind the virus particles when discharged from the mouth and/or nose of an infected individual. The virus binding molecules can be, for example, antibodies. The present disclosure may refer to antibodies as the virus binding molecules but it will be understood that other virus binding molecules may also be used and are within the scope of this description.

The term “test region” as referred to herein relates to the region of the virus detection system wherein the viral particles are deposited for the detection of a viral infection. The viral particles may be deposited in the test region when the facemask is worn by an individual. The test region may include one or more components of the virus detection system, e.g. viral binding molecules, one or more detection reagents and the like. The viral particles may be transported from the test region to other regions of the virus detection system in order to interact with the other components of the virus detection systems, e.g. viral binding molecules, one or more detection reagents and the like. Alternatively, other components of the virus detection system, e.g. viral binding molecules, one or more detection reagents and the like may be transported to the test region with the discharged viral particles.

Unless otherwise indicated, the molecular biology, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), R. Scopes, Protein Purification Principals and Practice, 3^{rd} edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### DETAILED DESCRIPTION

The present description includes a facemask with an integrated virus detection system. The virus detection system can be embedded within a facemask worn by an individual susceptible to a viral infection. The individual can wear a facemask for protective purposes during work or leisure in a public and/or a private setting. If the individual develops a viral infection and begins discharging viral particles from mouth and/or nose, the virus detection system embedded in the facemask can detect the virus and provide a positive indicator signal indicative of viral infection. The facemasks described herein serve as a protective barrier to prevent discharge of the virus into the environment and also as a detection system for the presence of a virus in an individual. The virus detection system in the facemask can be configured to detect one or more viruses.

The present description also includes methods of detecting a viral infection in an individual by use of the facemasks described herein. The method can include providing a facemask with an integrated viral detection system. The individual can wear the facemask and conduct their daily tasks. Individuals that have the viral infection and have begun to discharge viral particles from their mouth and/or nose would accumulate viral particles in the virus detection system, e.g. in the test region, within the facemask. After removal of the facemask, the test region of the virus detection system in the facemask can be analyzed by one or more detection reagents. The detection system can provide a positive indicator signal if the individual wearing the facemask is infected with the virus, even with low viral loads. The present description also includes a method of conducting business by providing a facemask with an integrated virus detection system for use in a workplace, a public and/or a private setting.

In many viral diseases, the spread of the virus is greatest during the asymptomatic phase or the early symptomatic phase that is around and immediately following the onset of symptoms. Virus excretion is comparatively low during the initial phase of SARS-CoV-2. It peaks in respiratory specimens and in stools at around day 10 after the onset of the clinical illness. In order to make an early diagnosis, it is therefore necessary to use highly sensitive tests that are able to detect the low levels of the virus during the first days of the infection.

There are many non-standardized and sensitive tests under development in many countries. The available SARS RT-PCR based diagnostic tests often suffer the drawback of being complex and difficult to administer. The typical SARS diagnostic test uses nested (two step) polymerase chain reaction (PCR) to accomplish a certain level of specificity and sensitivity. The tests can be expensive, may have to be conducted by trained personnel and can deplete critical reagents. The results from the test can require the use of a device that is limited by the number of tests it can process in a day.

The present description relates to a wearable personal virus detection system that can be compatible with generating an indicator signal, e.g, a visual signal, when an individual is infected with one or more of the target viruses. Since the virus detection system is embedded or integrated within a protective facemask, the individual can wear the facemask for a variable length of time. Thus, even if the viral titers are low, the prolonged exposure of the test region to the discharged particles from the individual can enable detection of the virus. The virus detection system in the facemask may provide a positive indicator signal for the virus after the individual wears the facemask, for example, between about an hour and about two hours. Embedding the virus detection system within a facemask can advantageously allow the virus to accumulate on the test region over a prolonged period of time. The use of the facemask can allow detection of the virus in an individual in an early phase of the infection when an individual may be asymptomatic.

In one embodiment, the present description can include a facemask with a virus detection system. The facemasks described herein can be configured to cover the nose and mouth of an individual and act as a barrier for discharge of the viral particles into the environment. Facemasks are known in the personal protective equipment field and any of the facemasks known in the field can be configured to incorporate a virus detection system within the facemask.

In some embodiments, the virus detection system can be integrated in the facemask, By integrated it is meant, that the virus detection system may be permanently and/or temporarily embedded within the facemask and/or on a surface of the facemask. In one embodiment, the virus detection system may be removably incorporated into the facemask. In one embodiment, the inner surface of the facemask may serve as the virus detection system. In one embodiment, the facemask may include an opening, e.g. a slot, for insertion of a virus detection system. When the virus detection system, e.g. a test strip or a test device, is placed in the opening, a test region of the virus detection system can be sufficiently exposed to capture any discharged viral particles from the individual wearing the facemask. In one embodiment, the virus detection system may be removed for detection of the virus particles. In one embodiment, the presence of the viral particles can be detected while the virus detection system remains in the opening of the facemask. In one embodiment, a virus detection system can be constructed within the facemask with a protective cover over the test region. When an individual is ready to use the facemask, the protective cover over the test region can be removed prior to use. It will be understood that there are other embodiments for integrating a virus detection system within a facemask and these embodiments are all within the scope of this description.

The virus detection system can be integrated into the facemask in a variety of configurations. In one embodiment, the virus detection system may be affixed to the facemask in a manner that the test region of the virus detection system can capture the expelled respiratory droplets and/or aerosolized particles from the individual. The detection system may be affixed, for example, on the inner surface of the facemask. In one embodiment, the facemask may have an opening or a window that is configured to retain a virus detection system. In one embodiment, all or a portion of the inner surface of the facemask may serve as the virus detection system. Other configurations of integrating a virus detection system within a facemask are also included and all are within the scope of this description.

The virus detection system in the facemask can be accessible to receive the discharge from an individual's mouth and/or nose. If the individual is infected with a virus, the individual can discharge virus particles that would be absorbed and/or retained by the virus detection system within the facemask. The discharged virus particles may be respiratory droplets and/or aerosolized particles from an individual with a viral infection. The discharge may also be a liquid from the individual such as saliva. After sufficient time has elapsed, the virus detection system in the facemask can be evaluated for the presence of viral particles.

The facemasks can be made from a variety of materials. Facemasks can be N95 masks or N95-type masks, surgical masks, cloth masks or other masks that are compatible with an individual wearing a facemask for an extended period of time. In some embodiments, the facemasks may include, for example, polypropylene filter cloth, nylon cloth, woven polyester cloth, nonwoven polyester cloth and the like. All or a portion of the facemasks may also include cotton materials or materials derived from cotton. Facemasks with other materials are also within the scope of this description. In one embodiment, the material for the facemask may retain respiratory droplets or aerosolized particles that include viral particles. In one embodiment, the facemask may include materials that minimize the release of viral particles into the atmosphere. The facemasks may be retained close to the nose and/or mouth of the individual by ties, elastic, wires and the like.

The virus detection system integrated into a facemask can detect one or more of a variety of viruses. The viruses that may be detected can include viral particles that are released within respiratory droplets, saliva and/or aerosolized particles from an individual harboring the virus. The virus detection system can detect DNA viruses and/or RNA viruses. In some embodiments, the viruses can be, for example, influenza viruses, Coronavirus. Henipavirus, Ebola virus, Hantaan virus, Lassa fever virus, Marburg virus, Crimean-Congo haemorrhagic fever virus, Monkeypox virus, Rift Valley Fever virus, South American haemorrhagic fever viruses, Central European tick-borne encephalitis virus, Far Eastern tick-borne encephalitis virus, Japanese encephalitis virus, Russian swing and summer encephalitis virus, Kyasanur forest disease virus, Omsk hemorrhagic fever virus and West Nile virus. In some embodiments, the viruses detected by the virus detection system can also include RNA viruses of the order Mononegavirales that contain single-stranded genomes that are negative sense. These viruses can include, for example, Orthomyxoviridae (which contains the influenza viruses) and Paramyxoviridae (which contains the parainfluenza viruses (PIVs), human respiratory syncytial virus (RSV), and human metapneumovirus (hMPV). The virus detected by the virus detection system can also include, for example, Picornaviridae, rhinoviruses, enteroviruses (such as coxsackieviruses and numbered enteroviruses). In some embodiments, DNA viruses such as Adenoviridae, Parvoviridae and the like can also be detected.

In some embodiments, the viruses detected by the virus detection system can include, for example, viruses from the family Coronaviridae, an enveloped, positive-sense single-stranded RNA (ssRNA). These viruses can include, for example, human coronavirus (HCoV) 229E, HCoV OC43, the severe acute respiratory syndrome-associated CoV (SARS-CoV), Middle East respiratory syndrome-related coronavirus (MFRS-CoV), HCoV, NL63, HCoV HKU1 and the like. In one embodiment, virus detections system in facemasks can detect SARS-CoV-1 and/or SARS-CoV-2.

The virus detection system can include a solid support. The solid support can include a variety of materials known in the art and compatible for attachment of a virus binding molecule, e.g. an antibody. Attachment of the virus binding molecules to the solid support can be through a variety of methods and can include, for example, absorption, adsorption, covalent attachment and the like. In some embodiments, the solid support may include one or layers. In some embodiments, the solid support may include particles, e.g. nanoparticles, and/or other solid support materials to retain the particles. Each of the layers may include same materials and/or different materials. In one embodiment, the solid support may be a nitrocellulose membrane or other suitable membrane. In some embodiments, the support may also be fibers, fiberglass, latex or a plastic material such as polystyrene or polyvinylchloride. In some embodiments, the solid support can include polypropylene filter cloth, nylon cloth, woven polyester cloth, nonwoven polyester cloth, cellulose acetate membrane, cellulose nitrate membrane, polyethylene filter, analytical paper, nitrocellulose membranes. The solid support may also include compartments, channels, e.g. fluidic channels. The solid support may be other materials that are compatible for attachment and/or retention of some or all of the components of the virus detection system and all are within the scope of this description.

In some embodiments, the virus detection system may also include particles as a solid support and/or for attachment of the viral binding molecules, one or more detection reagents and/or for other components of the virus detection system. The particles can include, for example, agarose particles, polystyrene particles, cellulose particles, polyacrylamide particles, latex particles, magnetic particles, nanoparticles and the like. Examples of commercially available matrices include, for example, Sepharose® (Pharmacia), Poros® resins (Roche Molecular Biochemicals), Actigel Superflow™ resins (Sterogene Bioseparations Inc.), and Dynabeads™ (Dynal Inc.). The selection of the particles can vary and may depend on such features as stability, capacity, accessibility of the coupled antibody, flow rate (or the ability to disperse the resin in the reaction mixture), ease of separation, detection reagents, indicator signals and the like.

The virus detection system can include a test region in the solid support. The test region may include one or more components of the virus detection system. In some embodiments, the test region can include one or more viral binding molecules, e.g. antibodies, for capturing/binding the viral particles discharged by an infected individual. In some embodiment, respiratory droplets and/or aerosolized particles containing the viral particles can be discharged from the individual onto the test region. The antibodies at the test region can bind and/or capture the viral particles. In some embodiments, the discharged particles can be first deposited on the test region and then the viral binding molecules and/or the one or more detection reagents are placed, transported or provided to the test region. In some embodiments, the discharged particles from the individual can be deposited on the test region and upon initiation of the assay, the discharged particles, if present, are transported from the test region to the other regions of the virus detection system. In other words, the sample to be analyzed is placed in the test region and transported to a detection region of the virus detection system. In some embodiments, the test region and the detection region are at the same location in the virus detection system. In some embodiments, the test region and the detection region are at a different location in the virus detection system.

Purified, recombinant or synthesized antigens can be prepared to generate antibodies to the viral particles by any of a variety of techniques known to those of ordinary skill in the art. See, e.g., Harlow and Lane, Antibodies: A Laboratory Manual, Cold Spring Harbor Laboratory, 1988. In one such technique, an immunogen comprising the antigenic polypeptide is initially injected into any of a wide variety of mammals (e.g., chickens, mice, rats, rabbits, sheep and goats). In some embodiments, the antigen, such as the SARS-CoV-2, may serve as the immunogen. A variety of methods to generate antibodies to viruses are known and may be used. In one embodiment, a method described in, for example, U.S. Pat. No. 9,849,175 to Mitteness may be used and is incorporated herein by reference in its entirety. In some embodiments, an immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the antigen may then be purified, for example, affinity chromatography using the antigen coupled to a suitable solid support.

In one embodiment, the spike protein from SARS-CoV-2 can be used as the antigen to generated antibodies to be used in the virus detection system. The spike protein can include two subunits and antibodies may be generated against subunit 1 (S1), subunit 2 (S2) or both subunits. All or a portion of the spike protein may be used to immunize, for example, chickens. All or portion of S1 and/or S2 may be used as immunogens to generate avian antibodies. The spike protein may be isolated from the viral particles or the spike protein may be expressed in a recombinant expression system. In one embodiment, recombinant spike protein is isolated and used as an immunogen to administer to chickens to generate avian antibodies.

In one embodiment, avian antibodies generated against the spike protein of SARS-CoV-2 can be used in the virus detection system. The avian antibodies include IgY antibodies. The avian antibodies may also be generated against other antigens from SARS-CoV-2 or the whole SARS-CoV-2 viral particles. The SARS-CoV-2 particles may be inactivated viral particles. The avian antibodies may be from chickens, geese, ostrich, duck and/or other avians.

In some embodiments, the virus detection system can be configured as an immunoassay. In some embodiments, the virus detection system can be configured as an enzyme immunoassay (EIA), an enzyme-linked immunosorbent assay (ELISA) system, a lateral flow immunochromatographic assay (LFIA), a three-dimensional paper-based assay and the like for detection of an antigen, e.g. SARS-CoV-2. Other types and formats of binding assays are known and may be configured for virus detection systems within facemasks and are within the scope of this description. It will be understood the present description will refer to enzyme immunoassays with color detection but other assay formats compatible with other detection methods such as chemiluminescent detection, fluorescence detection, thermal detection and the like are also within the scope of this description.

In some embodiments, EIAs in any of the formats known can be configured for integration as a viral detection system into a facemask. In one embodiment, this assay can be performed by first immobilizing an antibody (referred to as the capture antibody) on a solid support, e.g. a nitrocellulose membrane. The immobilized antibody may then be exposed to the biological sample, e.g. antigen such as SARS-CoV-2 (if individual is infected) and allowed to bind to the antigen, if present in the sample, to form an antibody-antigen complex or conjugate. In general, the test region can capture the viral particles over some or all of the duration when the facemask is worn by an individual. An infected individual, even in the early stages of infection, will discharge viral particles sufficient to provide a detectable signal in the virus detection system. It will be understood that the binding affinity between the antibody and the viral particles can be high enough to capture the expelled viral particles.

In some embodiments, detection reagents may be added to the virus detection systems after removal of the facemask. In some embodiments, the detection reagents can be included within the virus detection systems prior to the use of the facemask. Detection reagents can include any compound that can bind to the immobilized antibody-antigen complex and that can be detected by any of a variety of means known to those in the art. The detection reagents can contain a binding agent (such as, for example, Protein A, Protein G, immunoglobulins, lectin or an antibody) conjugated to a reporter group. Detection reagents can also include reporter groups. Reporter groups can include enzymes (such as horseradish peroxidase, alkaline phosphatase, catalase and the like), substrates, cofactors, inhibitors, dyes, radionuclides, luminescent groups, fluorescent groups and other detection molecules such as biotin, avidin, streptavidin and the like. The conjugation of a binding agent to a reporter group may be achieved using standard methods known to those of ordinary skill in the art. Common binding agents may also be purchased conjugated to a variety of reporter groups from many sources (e.g., Zymed Laboratories, San Francisco, Calif. and. Pierce, Rockford, Ill.).

The one or more detection reagents can be incubated with antibody-viral complex for an amount of time sufficient to detect the bound antigen. In some embodiments, the antibody-viral complex may be immobilized. An appropriate amount of time may generally be determined from the manufacturer's instructions or by assaying the level of binding that occurs over a period of time. In some embodiments, unbound detection reagent may then be separated and the bound detection reagent may be detected using, for example, a substrate that reacts with the reporter group retained in the test region that can result in a positive detection signal if an individual is infected with the target virus or viruses. The method employed for detecting the reporter group depends upon the nature of the reporter group. In one embodiment, the detection of reporter group is a colorimetric detection. In one embodiment, the detection can be a thermal detection and can include components needed for thermal detection. Other detection methods may also be used and all are within the scope of this description.

In one exemplary embodiment, the detection reagents can include a second antibody attached to a reporter group, e.g. alkaline phosphatase, horseradish peroxidase, and the like. The second antibody with the attached reporter group can bind to any viral antigen/antibody complex. The detection reagents can also include a substrate that can be acted upon by any bound reporter group on the second antibody to produce a indicator signal. The enzymatic action of the reporter group present on the substrate can correlate with the presence or detection of the virus in the test/detection region. A facemask worn by an infected individual can lead to a positive indicator signal in the detection/test region.

The virus detection system can be configured for a variety of assay formats. The assay may be a direct assay or an indirect assay. The virus detection system can be a sandwich assay, a competitive assay, and the like. A variety of assay formats are known in the field and all are within the scope of this description.

In some embodiments, the assay in the virus detections system may be initiated by the addition of a liquid. The liquid can be one of the components of the virus detection system, e.g. one or more of the detection reagents. In some embodiments, the assay may be initiated by the additions of a liquid such as water, buffer, saliva and the like. In some embodiments, the assay in the virus detection system may be initiated as the discharge of the viral particle occurs into the test region of the virus detection system.

In some embodiments, the virus detection system may be configured to detect more than one virus. The test region, for example, can include a number of dots or bands and each of the dots and/or bands can be configured to assay a different virus. The individual may be screened for multiple viruses simultaneously or separately.

In some embodiments, the virus detection system may be a lateral flow assay system. The facemask, for example, may have a test strip within the facemask. After removal of the facemask, the test strip may be removed and one or more detecting reagents, buffer, water and/or saliva may be placed on the test strip to initiate the detection process. In one embodiment, the test strip may include all the necessary detection reagents and the detection process may be initiated by the placement of water and/or other liquid at an initiation region. The addition of the liquid, initiates the reaction and a lateral flow assay can be conducted to reveal if viral particles are present. In one embodiment, the presence of viral particles can be a colorimetric determination, e.g. test/detection region turns blue if viral particles are present. The test strip can include other binding molecules, reporter molecules, substrates and the like needed for detection of an analyte. This embodiment can be similar to a pregnancy test or dipstick type tests for the detection of an analyte.

In some embodiments, the virus detection system can be a multilayer detection system or a three-dimensional detection system, A variety of multilayer detection systems are known in the art and are within the scope of this description. In some embodiments, the multilayer detection systems can be based on systems as described in Fernandes et al., Journals of Visualized Experiments, Issue 121, March 2017, Page 1-10, or as described in US Patent Publication No. 2019/0049349 to Lee et al, both are incorporated herein by reference in their entirety.

In one embodiment, the virus detection system may be a vertical flow and/or a three-dimensional microfluidic device assay system. The facemask, for example, may have a test strip within the facemask. After removal of the facemask, the test strip may be removed and one or more detecting reagents, buffer and/or water may he placed on test strip to initiate the detection process. In one embodiment, the device may include all the necessary detection reagents and the detection process may be initiated by the placement of water, buffer, saliva and/or other liquid at an initiation region. The addition of the liquid, can initiate the reaction to reveal if viral particles were present in the test/detection region. In one embodiment, the presence of viral particles can be a colorimetric determination, e.g. test/detection region turns blue if viral particles are present. The device can include other binding molecules, reporter molecules, substrates and the like needed for detection of the viruses.

The present description also includes methods of diagnosing or detecting a viral infection in an individual. The method can include detecting the presence of a virus in an individual by the use of facemasks described herein. In one embodiment, a facemask can be appropriately placed over the face of an individual to cover the mouth and/or nose area. The facemask can serve as a barrier for discharge from mouth and/or nose of an individual. The status of the individual can vary and anyone can wear the mask. The facemasks described herein can be worn by individuals susceptible to viral infection, by individuals that are infected and asymptomatic and/or by individuals with symptoms. The facemasks may also be worn by individuals that have been diagnosed as positive for viral infection as a barrier. In some embodiments, the virus detection system may be configured to detect the level of infection, e.g. stronger color reaction with higher viral load. The virus detection system may be configured to detect low levels of viral loads, medium levels of viral loads and/or high level of viral loads.

In some embodiments, the facemasks are worn in a work setting, a public setting and/or a private setting. In one embodiment, the facemask may be worn for a part or an entire workday. During use of the facemask, an individual with a viral infection can discharge viral particles through the mouth and/or nose and these viral particles can be bound and/or trapped in the test region of the virus detection system. The virus detection system can then be evaluated for the presence of viral particles after a determined elapsed time and/or at the end of the workday. The integration of the virus detection system within the facemask is advantageous because the test region of the virus detection system can be exposed to the discharge from individuals over an extended period of time, if necessary. Without being bound by any theory, it is thought that the test region can accumulate the viral particles over the duration of facemask use, e.g. between 5 minutes and 10 hours, and thus may be able to detect viral infections in an individual even when the viral loads are low in the individual.

A facemask can be worn for various lengths of time by an individual prior to detecting a positive indicator signal in an infected individual. The facemasks may be worn, for example, for at least five minutes, or at least 30 minutes, or at least one hour, or at least two hours, or at least four hours, or at least six hours, or at least eight hours. In some embodiments, the facemask may be worn for at least one hour in order to detect a viral infection in an individual. In one embodiment, the facemask may be worn between about one hour and about twelve hours to detect a viral infection in an individual. Facemasks may be worn for a longer period of time and all are within the scope of this description.

The method can further include evaluation of the facemask for the presence of a viral infection by one or more viruses. The facemask can be removed from the user after a determined period to evaluate the presence of a viral infection in an individual. In one embodiment, the facemask is removed at the end of the workday for evaluation of viral infection. In one embodiment, the facemask is removed prior to the end of the workday for evaluation of viral infection, e.g. after about one or two hours.

The method can further include analyzing the test region for the presence of viral particles. In one embodiment, the method may further include addition of one or more detection reagents to the virus detection system. The detection reagents may all be added at the same time or in a stepwise manner. In some embodiments, one or more steps may be conducted by the addition of one or more detection reagents to detect the presence of viral particles in the virus detection system. In one embodiment, all of the components needed for detection are included in the test region and removal of the facemask may reveal a positive indicator signal, a color response. It will be understood that other embodiments for determining the presence of viral particles in a binding assay may be used and all are within the scope of this description.

The present description also includes a method of managing a public environment such as a workplace. The method can include providing a facemask with a viral detection system as described herein. The viral detection system in the facemask is evaluated for the presence of viral particles after a prescribed length of time and/or at the end of a workday. The method can lead to early detection of a viral infection. This can be particularly advantageous in workplace environments or public environments that may be congested or environments where people work for prolonged periods in constrained spaces.

Although the present invention has been described with reference to preferred embodiments, workers skilled in the art will recognize that changes may be made in form and detail without departing from the spirit and scope of the invention.

All publications, patents and patent documents are incorporated by reference herein, as though individually incorporated by reference, each in their entirety, as though individually incorporated by reference. In the case of any inconsistencies, the present disclosure, including any definitions therein, will prevail.

1. A facemask for an individual comprising a virus detection system, wherein the virus detection system comprises a test region and virus binding molecules, the test region positioned in the face mask to receive respiratory droplets and/or aerosol particles discharged from the individual, wherein the virus detection system is configured to detect the presence of one or more virus.

2. The facemask of claim 1, wherein the virus detection system further comprises one or more detection reagents to provide a signal when the virus is bound to the virus binding particles.

3. The facemask of claim 1, wherein the virus detection system is an EIA test.

4. The facemask of claim 1, wherein the virus binding molecules are antibodies.

5. The facemask of claim 1, wherein the virus binding molecules are avian antibodies.

6. (canceled)

7. (canceled)

8. The facemask of claim 1, wherein the virus is severe acute respiratory syndrome coronavirus-2 (SARS-CoV-2).

9. The facemask of claim 1, wherein the virus binding molecules bind the spike protein of SARS-CoV-2.

10. (canceled)

11. The facemask of claim 1, wherein the one or more detection reagents are selected from the group consisting of enzymes, reporter groups, substrates, cofactors, inhibitors, dyes, radionuclides luminescent groups, fluorescent groups, colorimetric indicators, thermal detection components.

12. (canceled)

13. A method of detecting a viral infection in an individual comprising analyzing the viral infection in the individual through a virus detection system embedded in a facemask, wherein the individual wears the facemask for a time period, wherein the test region is placed to receive the respiratory droplets and/or aerosol particles discharged from the individual, the virus detection system comprising a test region and virus binding molecules, wherein the virus detection system is configured to detect one or more viruses.

14. (canceled)

15. The method of claim 13, wherein the time period is between about five minutes and 10 hours.

16. (canceled)

17. The method of claim 13, wherein the individual wears the facemask during an activity.

18. The method of claim 13, wherein the test region provides a positive indicator signal when the individual is infected with the virus.

19. (canceled)

20. The method of claim 13, wherein the positive indicator signal is present upon addition of a substrate after the removal of the face mask if the individual is infected with the virus.

21. (canceled)

22. (canceled)

23. The method of claim 13, wherein the virus binding molecules are avian antibodies.

24. (canceled)

25. (canceled)

26. The method of claim 13, wherein the virus is severe acute respiratory syndrome coronavirus-2 (SARS-CoV-2).

27. (canceled)

28. The method of claim 13, wherein the virus binding molecules bind the spike protein of SARS-CoV-2.

29. (canceled)

30. (canceled)

31. A method of operating a workplace comprising providing a facemask comprising a viral detection system, wherein individuals working in the business wear the face mask during the work day and wherein the viral detection system in the facemask is evaluated for the presence of the virus after a time period, wherein the virus detection system is configured to detect one or more viruses.

32. The method of claim 31, wherein the business is a meat processing factory.

33. The method of claim 31, wherein the time period is between about 5 minutes and 12 hours.

34. The method of claim 31, wherein the detection system comprises a colorimetric signal.
